(19) United States
(12) Patent Application Publication
(10) Pub. No.: US 2019/0275289 A1
(43) Pub. Date: Sep. 12, 2019

(54) COMPACT URINARY CATHETER

(71) Applicant: COMPACTCATH, INC., Palo Alto, CA (US)

(72) Inventors: Daniel Wei-Chen HONG, Cupertino, CA (US); Naama Stauber BRECKLER, Palo Alto, CA (US)

(21) Appl. No.: 16/287,594

(22) Filed: Feb. 27, 2019

(30) Foreign Application Priority Data
Mar. 12, 2018 (CN) 201810201903.4
Apr. 2, 2018 (CN) 201810282802.4
Apr. 3, 2018 (CN) 201820454903.0

(51) Int. Cl. A61M 25/00; A61F 5/44

(52) U.S. Cl. CPC A61M 25/0017; A61M 2202/0496; A61F 5/44; A61M 25/002

(57) ABSTRACT

A urinary catheter system includes a urinary catheter, a slidable sheath surrounding a portion of the catheter proximate the distal end, and an enclosure having a bottom member and a top member that rotate relative to one another about an axis of rotation. Rotating the top member or the bottom member relative to the other member causes the catheter to wind into a spiral between the inner wall of the bottom member and the outer wall of the top member. Some embodiments also include a urine collection bag attached to the proximal end of the catheter and housed within an inner open portion of the enclosure. Some embodiments also include a hygienic sleeve disposed over at least part of the catheter, to enhance cleanliness of the catheter before use.

### CROSS-REFERENCE TO RELATED APPLICATIONS

This application claims the benefit of Chinese Patent Application No. 201810201903.4, filed Mar. 12, 2018, and Chinese Patent Application No. 201810282802.4, filed Apr. 2, 2018, and Chinese Patent Application No. 201820454903.0, filed Apr. 3, 2018. The disclosure of these priority applications is hereby incorporated by reference in its entirety into the present application.

### TECHNICAL FIELD

This application is generally related medical devices, systems and methods. More specifically, the application is related to urinary catheters, assemblies including urinary catheters, and medical uses thereof.

### BACKGROUND

Some patients require urinary catheters to void their bladders, such as those patients who suffer from urinary incontinence or other medical disorders. Urinary catheters are inserted into the bladder through the urethra, to open the urinary sphincter and drain urine into a toilet, urinary bag or other appropriate receptacle. A typical urinary catheter patient might void his or her bladder every two to four hours. In developed countries, urinary catheters are disposable (in other words, they are not reused by the patient). Thus, a patient typically uses multiple urinary catheters each day.

Using urinary catheters to void the bladder involves a number of different challenges. It can be difficult, for example, for the patient to use urinary catheters effectively and discretely, without social embarrassment and inconvenience. Catheters can be large and cumbersome, such that carrying and using multiple catheters during the activities of daily living can be inconvenient. It is often difficult for the patient to carry and conceal urinary catheters in bags or under clothing.

Similarly, urinary catheters typically need to be lubricated for insertion into the urethra. They can be pre-lubricated with a water activated coating or designed to be lubricated with a gel after opening. Either way, these lubricating mechanisms can be inconvenient, as users must either deal with packaging fluid, or carry around a lubricant along with the urinary catheter.

Maintaining proper hygiene of a urinary catheter can also be challenging, and poor catheter hygiene can lead to urinary tract and bladder infections. The difficulty in maintaining proper catheter hygiene typically arises from the catheter insertion process, during which the patient touches the catheter with his or her fingers. The catheter may also come in contact with other objects (clothes, etc.) that are not clean.

Finally, catheter disposal after use can be a challenge. Although catheters may sometimes simply be thrown in the trash, again this can sometimes lead to embarrassing situations and/or unclean disposal of a urine-covered catheter.

All of these difficulties in using urinary catheters lead some patients to not use them as often as they should, thus voiding less frequently and risking injury to, or malfunction of, the bladder and/or kidneys. Even when used frequently enough, the catheters still hold a risk of infection, for the reasons discussed above. Additionally, patients suffer from frequent embarrassment and inconvenience due to the carrying, use and disposal of their urinary catheters.

The assignee of the present application has developed a compact urinary catheter device, system and method of use to address at least some of the challenges of conventional urinary catheters. The device, system and method is described in U.S. Pat. Nos. 8,556,884, 8,708,999 and 8,974,438, as well as U.S. Patent Application Publication Number 2016/0001037, all of which are incorporated fully herein by reference. Although advancements have been made, continued improvements in urinary catheters are still desirable.

### BRIEF SUMMARY

In one aspect of the present application, a urinary catheter system, includes a urinary catheter having a distal end configured for insertion into a urethra and a proximal end, a sheath slidably disposed over a portion of the catheter near the distal end, an enclosure for housing the urinary catheter, and a urine collection bag attached to the proximal end of the catheter and housed within an inner open portion of the enclosure. The enclosure includes a bottom member having a base and an inner wall with an inner hole leading to an inner open portion of the enclosure and a top member having an outer wall with an outer hole leading out of the enclosure. The top member is attached to the bottom member so as to rotate relative to the bottom member about an axis of rotation, and rotating the top member or the bottom member relative to the other member causes the catheter to wind into a spiral between the inner wall of the bottom member and the outer wall of the top member.

In some embodiments, an outer diameter of the enclosure is no greater than 60 millimeters. In some embodiments, the inner wall of the bottom member of the enclosure includes multiple curved, vertical slats, and at least some of the vertical slats include a snap lock member for locking with a corresponding feature of the top member. In some embodiments, two of the vertical slats have a different curvature than the other vertical slats, to form the inner hole in the inner wall. The outer hole in the outer wall of the top member of the enclosure may be formed by an outer edge portion that is flexible to allow for pressing by the user to stop movement of the catheter into or out of the outer hole and an inner edge portion formed as a ramp to facilitate advancement of the catheter out of the outer hole.

Optionally, the system may also include a tubular adapter coupled between the urine collection bag and the proximal end of the catheter. The tubular adapter allows the urine collection bag to be removed from the proximal end of the catheter and allows a new urine collection bag to be attached to the catheter. In some embodiments, the sheath includes at least one outward facing protrusion for facilitating moving the sheath along the catheter by a user. Alternatively or additionally, the sheath may include at least one inward facing protrusion for facilitating moving the sheath along the catheter by a user.

Another optional component of the system is a hygienic sleeve disposed over at least a portion of the catheter to prevent contamination of the catheter before and during insertion into the urethra. For example, in some embodiments, the hygienic sleeve extends from the proximal end of the catheter to a proximal end of the sheath. In other embodiments, the hygienic sleeve extends from the proximal end of the catheter, over the sheath, and over the distal end of the catheter. The hygienic sleeve may be made of any suitable material, such as but not limited to any suitable plastic or polymer.

In some embodiments, the enclosure is a disposable package, and the catheter, the sheath and the urine collection bag are completely removable from the enclosure. In some embodiments, the enclosure is a teardrop-shaped package.

In another aspect of the present disclosure, a method for using a urinary catheter system to void a bladder of a patient may first involve contacting, with one of the patient's hands, a sheath on a catheter near a distal end of the catheter, where a majority of the catheter is housed in an enclosure including a top member and a bottom member, and where the distal end of the catheter is located outside an outer hole in the enclosure. Next, the method involves advancing the catheter out of the enclosure by pulling on the catheter via the sheath, sliding the sheath proximally along the catheter, and advancing the distal end of the catheter through a urethra of the subject into the subject's bladder. The method also involves unfolding a urine collection bag attached to a proximal end of the catheter, where the urine collection bag is stored in an inner open portion of the enclosure before unfolding. Finally, the method involves voiding the bladder through the catheter, so that urine passes from the bladder of the subject into the distal end of the catheter, out of the proximal end of the catheter and into the urine collection bag.

Optionally, the method may further involve, after voiding the bladder, removing the urine collection bag from the catheter, and winding the catheter at least partially into the enclosure by twisting a top portion of the enclosure or a bottom portion of the enclosure. The method may optionally further involve attaching a new urine collection bag to the proximal end of the catheter via a tubular adapter coupled between the proximal end of the catheter and the new urine collection bag.

In some embodiments, the method involves removing the catheter and the urine bag completely from the enclosure. In such embodiments, the method may also involve disposing of the catheter, the enclosure and the urine bag after the bladder is voided.

Contacting the sheath may involve contacting at least one outward facing protrusion on the sheath. Sliding the sheath proximally along the catheter may be performed multiple times, as the catheter is advanced through the urethra. In some embodiments, the method further includes sliding a hygienic sleeve proximally along the catheter during advancement of the catheter through the urethra.

These and other aspects and embodiments are described further below, in reference to the attached drawing figures.

### BRIEF DESCRIPTION OF THE FIGURES

FIG. 1 is a top perspective view of a urinary catheter and enclosure system, according to one embodiment;

FIG. 2 is a top, partially transparent view of the urinary catheter and enclosure system of FIG. 1;

FIG. 3 is a top perspective exploded view of the urinary catheter and enclosure system of FIGS. 1 and 2;

FIGS. 4A and 4B are top perspective views of the urinary catheter and enclosure system of FIGS. 1-3, illustrating retraction (FIG. 4A) and advancement (FIG. 4B) of the catheter into and out of the enclosure;

FIG. 5 is a top view of a urine collection bag of the system of FIGS. 1-4B;

FIG. 6 is a top perspective view of an enclosure of the system of FIGS. 1-4B;

FIG. 7A is an exploded view of the enclosure of FIG. 6;

FIG. 7B is a perspective view of a top portion of the enclosure of FIG. 6;

FIG. 7C is a perspective view of a bottom portion of the enclosure of FIG. 6;

FIG. 8 is a perspective view of a catheter, sheath and adapter of the urinary catheter and enclosure system of FIGS. 1-4B;

FIG. 9 is an exploded view of the catheter, sheath and adapter of FIG. 8;

FIG. 10 is a perspective view of a distal portion of the catheter and sheath of FIG. 8;

FIG. 11 is a side view of the catheter and sheath of FIG. 8, illustrating a method of use of the catheter and sheath, according to one embodiment;

FIG. 12 is a perspective view of a catheter, sheath and adapter of the urinary catheter and enclosure system of FIGS. 1-4B, with an optional hygienic sleeve disposed over the catheter, according to one embodiment;

FIG. 13 is a side view of the catheter, sheath and sleeve of FIG. 12, illustrating a method of use of the catheter, sheath and sleeve, according to one embodiment;

FIG. 14 is a perspective view of a distal portion of a urinary catheter and a sheath with an outward facing protrusion, according to an alternative embodiment;

FIG. 15 is a perspective view of a distal portion of a urinary catheter and a sheath with an outward facing protrusion, according to another alternative embodiment;

FIG. 16 is a perspective view of a distal portion of a urinary catheter and a sheath with an outward facing protrusion, according to another alternative embodiment;

FIG. 17 is a perspective view of a distal portion of a urinary catheter and a sheath with an outward facing protrusion, according to another alternative embodiment;

FIG. 18 is a perspective view of a distal portion of a urinary catheter and a sheath with an outward facing protrusion, according to another alternative embodiment;

FIG. 19 is a perspective view of a catheter and adapter of a urinary catheter and enclosure system, with an optional hygienic sleeve disposed over the catheter, according to one embodiment;

FIG. 20 is a perspective view of the catheter and adapter of FIG. 20, partially housed in the enclosure of the urinary catheter and enclosure system, according to one embodiment;

FIG. 21 is an exploded view of the system of FIG. 20;

FIG. 22 is a side view of the system of FIGS. 20 and 21, illustrating a method of use of the system, according to one embodiment;

FIG. 23 is a perspective view of a distal portion of a catheter, sheath and hygienic sleeve of a urinary catheter and enclosure system, according to one embodiment;

FIG. 24 is a perspective view of the complete system with the catheter, sheath and hygienic sleeve of FIG. 23, plus an enclosure;

FIG. 25 is a side view of the system of FIG. 24, illustrating a method of use of the system, according to one embodiment;

FIG. 26 is a perspective view of a distal portion of a catheter, sheath and hygienic sleeve of a urinary catheter and enclosure system, according to an alternative embodiment;

FIG. 27 is a perspective view of a urinary catheter and enclosure system, excluding some components of the system to highlight an adapter that connects the proximal end of a urinary catheter to a urine collection bag, according to one embodiment; and

FIGS. 28A-28C are various views of a package for a urinary catheter and enclosure system, according to one embodiment.

### DETAILED DESCRIPTION

Urinary catheter systems described herein generally include a disposable urinary catheter, a packaging device for the catheter, and in some embodiments a urinary collection bag and/or a hygienic sleeve for the catheter. These components of the system work together to provide a portable, easy-to-use, unobtrusive urinary catheter solution for patients. In some embodiments, the compact catheter system may include a catheter, packaging and optionally one or more other components, such as a urinary collection bag, a hygienic sleeve for the catheter, one or more lubricants, wipes and/or the like. In other embodiments, the system may include only a catheter and a package. In yet other embodiments, the system may include only a packaging unit and not a catheter, in which case, for example, the packaging unit may be used with one of a number of available catheters. Therefore, although the phrase “urinary catheter system” is used herein to describe various embodiments, this phrase should not be interpreted as limiting the system to a specific combination of components or devices.

This application describes embodiments of a urinary catheter system that provide users with discreet, hygienic, easy to use access to disposable urinary catheters. In some embodiments, the urinary catheter systems are so small, users can carry around multiple urinary catheters discretely and comfortably in a purse, backpack, briefcase or even pockets of clothing, without the inconvenience, bulkiness and hygiene risks associated with most currently available urinary catheters.

Unlike currently available urinary catheter packaging, some of the embodiments described herein include packaging that remains attached to the disposable urinary catheter during use. In use, the free end of the catheter is inserted into the urethra (using the sheath to avoid touching the catheter), and the opposite end of the catheter remains in the packaging. When the user voids, urine passes through the opposite end of the catheter, the packaging and then into the toilet, urine collection bag or other collection receptacle.

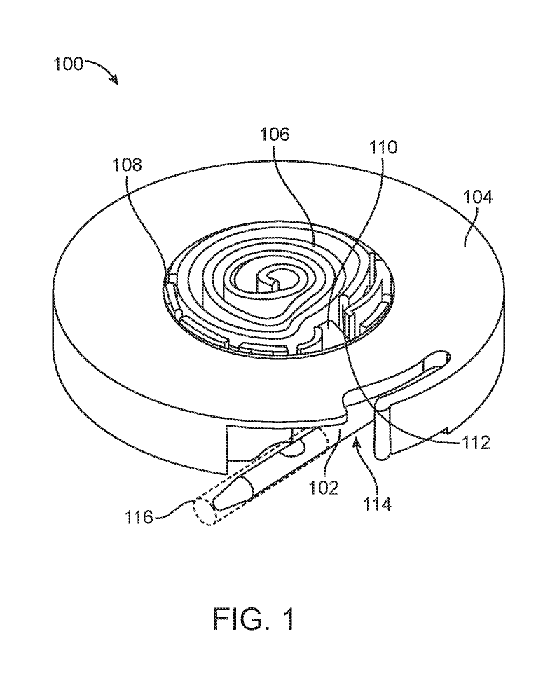

FIG. 1 is a top perspective view of one embodiment of a urinary catheter and enclosure system 100. The system 100 includes a urinary catheter 102, housed in an enclosure 104, and a urine collection bag 106 attached to a proximal end of the catheter 102 and housed inside of an inner wall 108 of the enclosure 104. (The area inside the inner wall 108 is referred to herein as the “inner open space” of the enclosure 104, as described further below.) The proximal end of the catheter 102 extends through an inner hole 112 in the enclosure 104 and attaches to the urine collection bag 106 via a tubular adapter 110, which is described further below. The distal end of the catheter 102 extends out of an outer hole 114 in the enclosure. A sliding sheath 116 is disposed over a distal portion of the catheter 102 and may cover the distal end or may be located just proximal to the distal end, according to various embodiments. Thus, the enclosure 104 has a donut-shaped configuration, within which the catheter 102 is wound. The collection bag 106 is disposed in a spiral shape within the inside portion of the enclosure, within the inner wall 108. (In alternative embodiments, the collection bag 106 may be folded in other patterns.) The outer diameter of the enclosure 104 (and thus the overall size of the system 102, when assembled), may be small enough to fit in the palm of an average user's hand. For example, in one embodiment, the outer diameter of the enclosure 104, might be less than or equal to about 60 millimeters (such as 58 millimeters in one embodiment), and the height of the enclosure 104 (or what might also be called its thickness) might be less than or equal to about 10 millimeters.

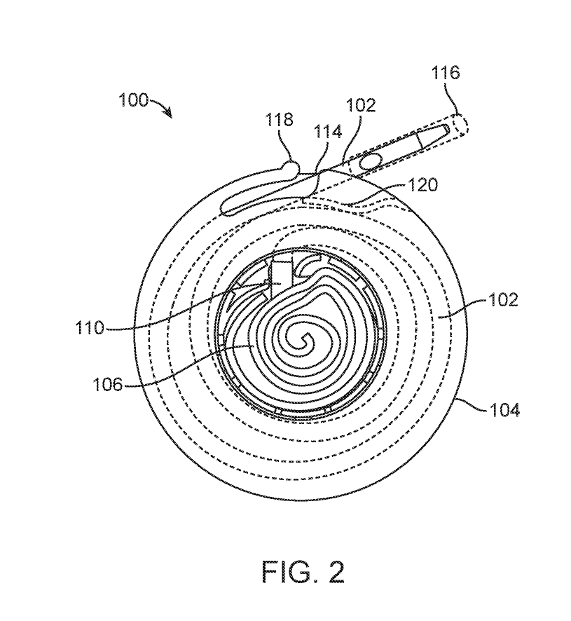

FIG. 2 is a top view of the urinary catheter and enclosure system 100 of FIG. 1, with the portion of the catheter 102 inside the enclosure 104 shown in dotted lines. As more clearly seen here, the proximal end of the catheter 102 is attached to the collection bag 106 via the tubular adapter 110. The outer hole 114 is formed by an outer edge portion 118 and an inner edge portion 120. In some embodiments, the outer edge portion 118 may be flexible, so that a user may press down on it to pinch the catheter 102 and thus stop it from further movement into or out of the enclosure 104 or in some cases even to stop the flow of urine through the catheter 102. The inner edge portion 120 may act as a ramp to help guide the catheter 102 out of the outer hole 114.

The sheath 116 is designed to slide proximally along the catheter 102, from its original, first position to a second position. The sheath 116 is used by the patient user to advance the catheter 102 into and through the urethra without the patient needing to actually touch the catheter 102 itself. The sheath 116 may be moved proximally along the catheter 102 to third, fourth and other subsequent positions to further advance the catheter 102 into the urethra. To advance the catheter 102, the sheath 116 is grasped by the user and is used to pull the catheter 102 out of the outer hole 114. The user then slides the sheath 116 proximally along the catheter 102 to a new, more proximal position. The user may then pull the catheter 102 farther out of the enclosure 104, again by pulling on the sheath 116. This process may be repeated as often as necessary, and it may be performed using only one hand of the user. During use, there need not be any predefined “first position,” “second position” and so on, of the sheath 116. Instead, the sheath 116 may be moved proximally and used to advance the catheter 102 in any convenient fashion by the user. The “positions” are simply used herein to more easily describe movement of the sheath 116 relative to the catheter 102. In general, the slidable sheath 116 allows a user to advance the catheter 102 out of the enclosure and into the urethra without touching the catheter 102, thus reducing the risk of infection.

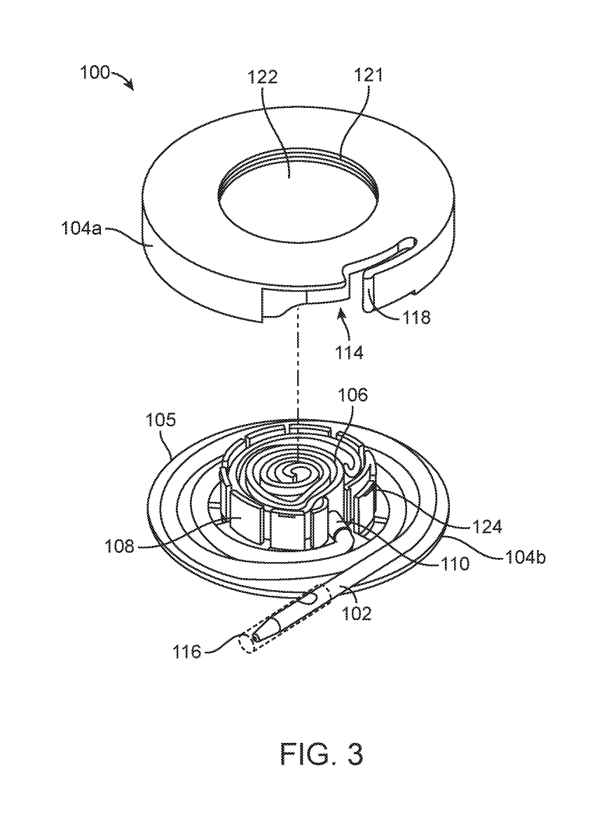

FIG. 3 is a partially exploded perspective view of the urinary catheter and enclosure system 100 of FIGS. 1 and 2. FIG. 3 illustrates that the enclosure 104 includes a top portion 104a (or “top shell”) and a bottom portion 104b (or “bottom shell”). The top portion 104a has an inner edge 121 that forms one corner of the inner open portion 122. The bottom portion 104b includes a flat base 105 and the inner wall 108, which in this embodiment is made up of multiple curved vertical slats that extend up from the base 105. The inner wall 108 forms the wall of the inner open portion 122 of the enclosure 104. Two of the slats have different curvatures than the other slats, to form the inner hole 112, through which the proximal end of the catheter 102 passes. Some or all of the slats that form the inner wall 108 may include a snap lock 124. The snap locks 124 allow the bottom portion 104b and the top portion 104a to snap-fit together. The catheter 102 winds around the inner wall 108 when it is wound into the enclosure 104. As noted above, the urine collection bag 106 is housed in the inner open portion 122 formed by the inner wall 108 and the inner edge 121, for easy, compact storage. It may be rolled in a spiral or otherwise folded, according to various embodiments.

In some embodiments, the top portion 104a and the bottom portion 104b are fixedly attached, so that the user will not disassemble the enclosure 104 during or after use. In these embodiments, the user might choose to wind the catheter 102 back up into the enclosure 104 after the user's bladder is voided, and the enclosure 104 and catheter 102 may then be disposed of or carried away to dispose of later. In an alternative embodiment, the top portion 104a and the bottom portion 104b may be easily removed from one another, to allow the catheter 102 (and urine collection bag 106, if included) to be completely removed from the enclosure 104 for use. In this type of embodiment, the enclosure 104 is thus acting merely as packaging for the catheter 102 and collection bag 106. In yet another embodiment, the bottom portion 104b may be provided alone, without any top portion 104a, and with the catheter 102 (and bag 106, if included) wrapped around it. In this type of embodiment, the bottom portion 104b, the catheter 102 and any other components of the system may be packaged inside of a sealed package, which, when opened, allows immediate access to the catheter 102. In yet other embodiments, the enclosure 104 may have an alternative shape, other than donut-shaped. For example, the enclosure 104 may be teardrop shaped in one embodiment. Thus, in general, the enclosure 104 may have many different forms and uses, according to various alternative embodiments.

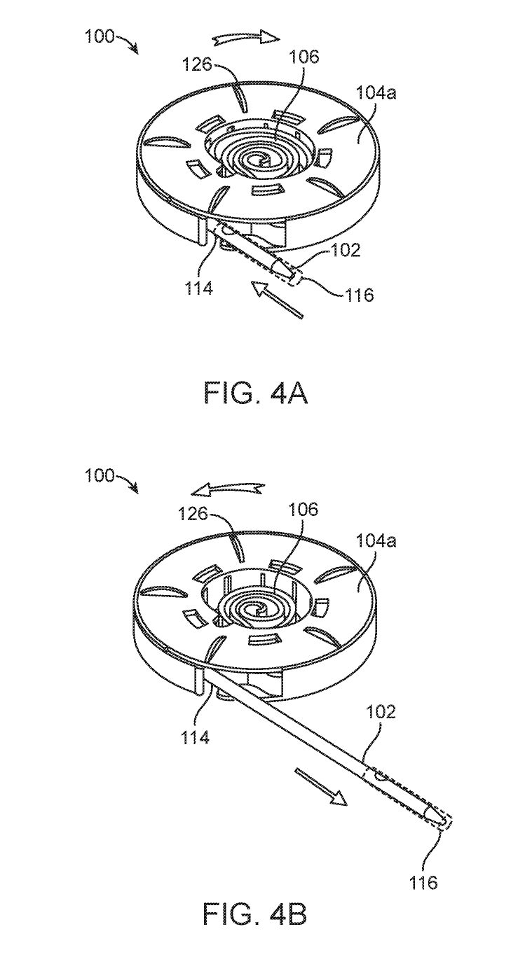

Referring now to FIGS. 4A and 4B, top perspective views of the urinary catheter and enclosure system 100 of FIGS. 1-3 are shown. In FIG. 4A, the catheter 102 is being retracted back into the enclosure 104 (hollow-tipped straight arrow), by winding the top portion 104a relative to the bottom portion 104b (hollow-tipped curved arrow). In various embodiments, either portion of the enclosure 104 may be wound relative to the other portion. In this embodiment, the top portion 104a of the enclosure 104 includes multiple surface features 126 (or “plectrums”) to help the user gain traction on the top portion 104a for winding and unwinding the catheter 102.

FIG. 4B simply illustrates advancement of the catheter 102 out of the outer hole 114, by winding the top portion 104a in the opposite (counter-clockwise) direction, compared to FIG. 4A. As mentioned above, the user may advance the catheter 102 in this direction simply by holding the sheath 116 and pulling the catheter 102 via the sheath 116. Once the catheter 102 is advanced into the urinary bladder, the urine collection bag 106 can be unfolded, to collect the urine flowing out of the catheter 102. Once voiding is complete, the bag 106 is removed from the proximal end of the catheter 102 and disposed of. Another bag 106 may be attached to the catheter 102 via the adapter 110, if the catheter 102 is to be used again. Alternatively, the adapter 110 may be used to attach a new urine collection bag 106 to a new catheter 102.

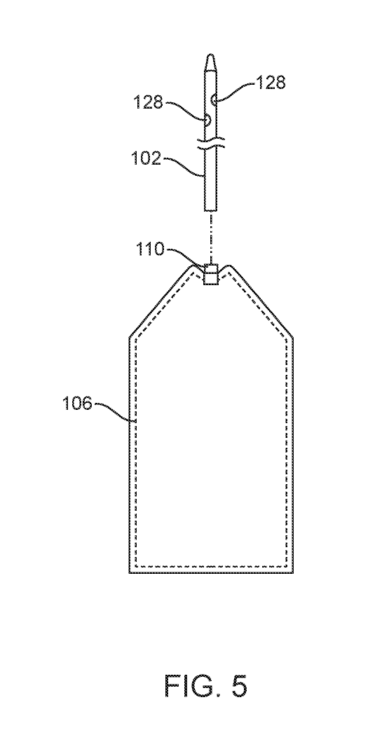

FIG. 5 is a top view of the urine collection bag 106 of the urinary catheter and enclosure system 100, shown in its flat, unwound configuration. As illustrated here, the catheter 102 is removable from, and attachable to, the collection bag 106 via the adapter 110. This is unlike conventional urine collection bags, which are permanently attached to a catheter. The catheter 102 further includes two urine draining apertures 128 (or other numbers in alternative embodiments) near its distal end. In various embodiments, the urine collection bag 106 may be folded into any suitable pattern or shape for housing inside of the inner open space 122 of the enclosure 104. For example, the bag 106 may be spiraled, folded back and forth on itself, twisted, scrunched into a ball or folded in any other suitable configuration. Also, the collection bag 106 may be made of any suitable material, such as but not limited to any suitable plastic or polymer.

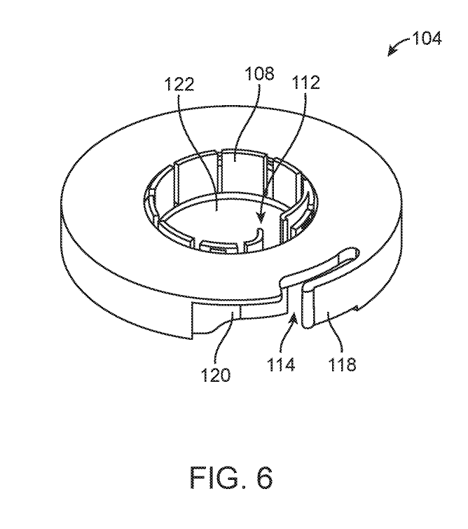

FIG. 6 is a top perspective view of the enclosure 104 by itself. As mentioned above, the outer edge 118 of the upper portion 104a of the enclosure 104 may be flexible in some embodiments. Outer edge 118 may also be considered a “handle,” which moves when it experiences pressure, for example, when it experiences handheld pressure by a user. Thus, the outer edge 118 may be used to hold the catheter 102 in place while the user slides the sheath 116 back (proximally) along the catheter body, thus preventing the catheter 102 from coiling back into the enclosure 104. Upon the user releasing this pressure from the outer edge 118, the catheter 102 is movable through the outer hole 114.

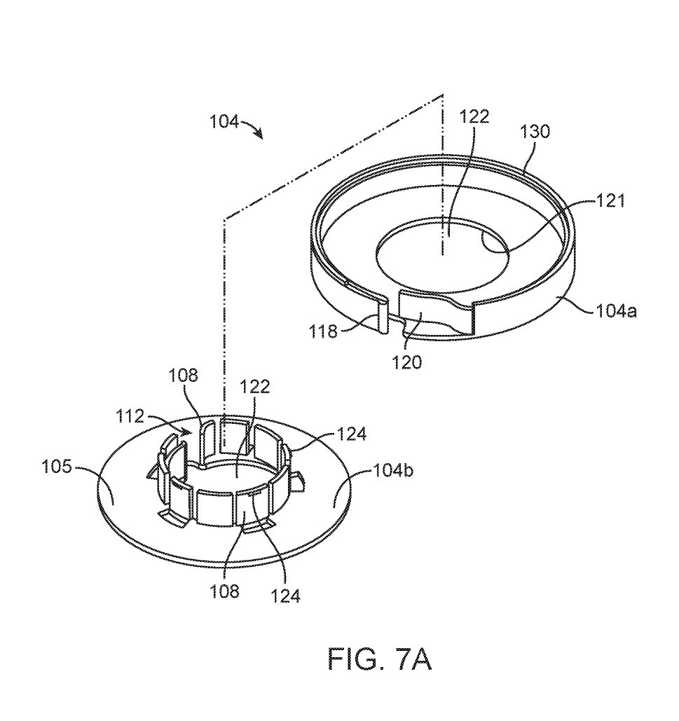

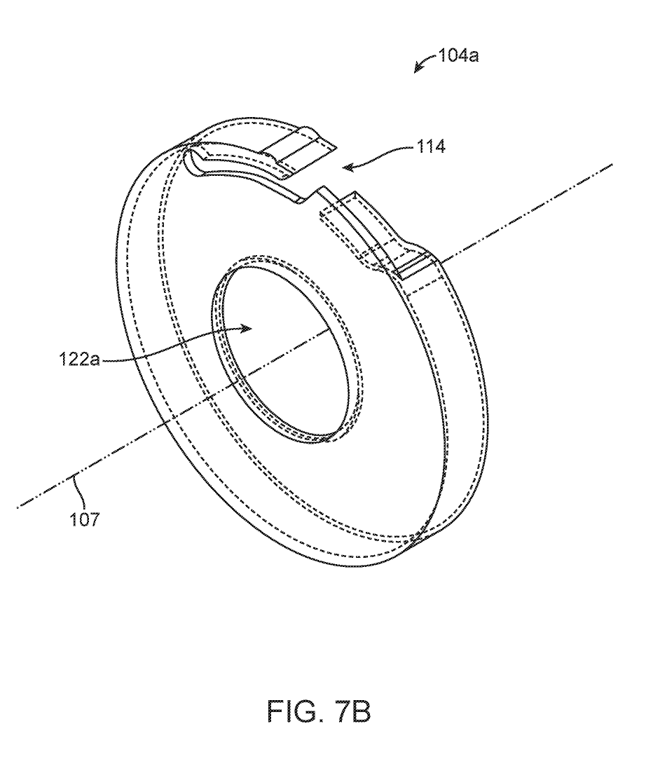

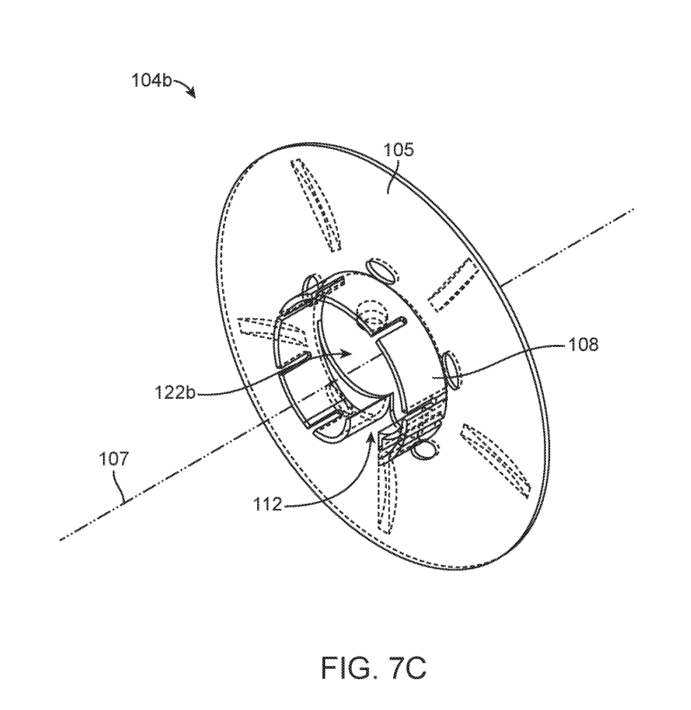

FIG. 7A is an exploded view of the enclosure 104, FIG. 7B is a perspective view of the top portion 104a, and FIG. 7C is a perspective view of the bottom portion 104b. FIG. 7A illustrates the multiple snap locks 124 on some of the curved, vertical slats of the inner wall 108 allow the top portion 104a to snap-lock down onto the bottom portion 104b. As mentioned previously, the inner edge 121 of the top portion 104a (see also FIG. 3) may have a groove, into which the snap locks 124 fit. Optionally, the bottom edge 130 of the top portion 104a may form a counter-bore, which wraps around or fits over the outer edge of the base of the bottom portion 104b when the enclosure is assembled. These features allow the top portion 104a and the bottom portion 104b to rotate smoothly relative to one another.

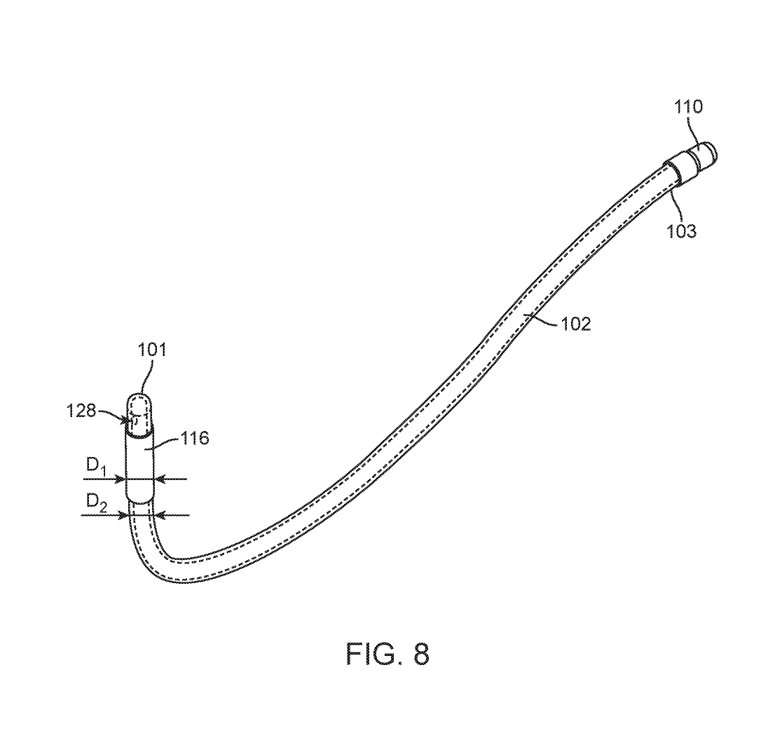

Referring now to FIGS. 8-18, a number of different aspects and embodiments of the sheath 116 of the urinary catheter and enclosure system 100 will be explained in greater detail. With reference to FIG. 8, as has been previously described, the catheter 102 of the system 100 has a distal end 101 and a proximal end. The catheter 102 may have any length and outer diameter suitable for passing through a urinary tract to access and drain a bladder, and the catheter 102 may be made of any suitable material, typically though not necessarily a flexible polymer. The adapter 110 fits onto the proximal end 103 of the catheter 102 and is used to connect the catheter 102 to the urine collection bag 106 (not shown). The adapter 110 may be made of any suitable material, such as but not limited to the same material used to make the catheter 102. Two drainage apertures 128 (or a different number in alternative embodiments) are located near the distal end of the catheter, for draining the urinary bladder. The sheath 116 is disposed over the catheter 102 and thus has a diameter D1 that is larger than the diameter D2 of the catheter 102. The sheath 116 is sized and configured to slide along the catheter 102 from the distal end 101 to the proximal end 103, and it may be made of any suitable material and may have any suitable length, thickness and outer diameter D1.

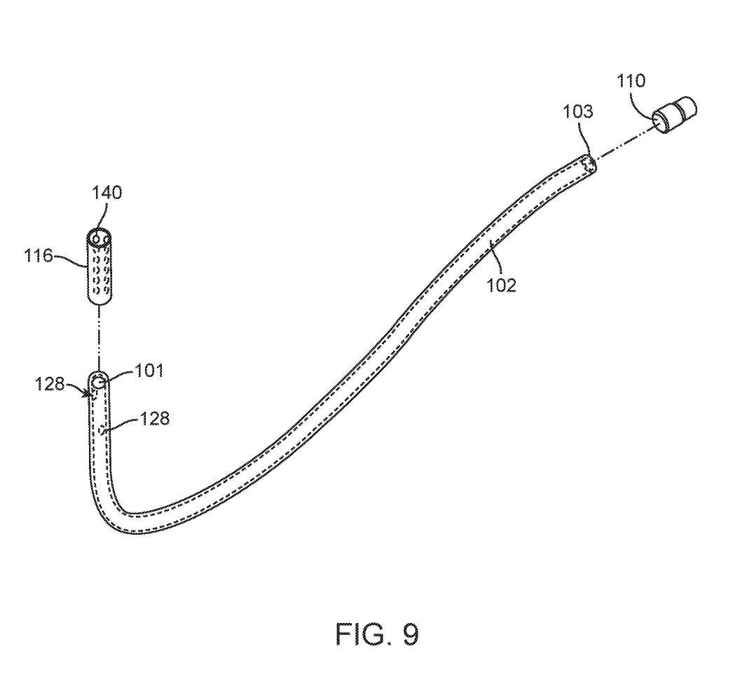

FIG. 9 is an exploded view of the embodiment of FIG. 8. In this figure, multiple bumps 140 (or “protrusions”) are illustrated on the inside surface of the sheath 116 as dotted lines. These bumps 140 may help to reduce friction as the sheath 116 slides along the outer surface of the catheter 102. This may be especially true when lubrication is used, as the bumps 140 create a small amount of space between the catheter 102 and the sheath 116, in the areas where there are no bumps 140. This allows lubricant to flow though those small spaces. At the same time, the surfaces of the bumps 140 may be roughened, for example by sanding or dull polishing, in order to prevent the bumps 140 from being so slippery as to increase the likelihood of the sheath 116 falling off the distal end 101 of the catheter 102. In various embodiments, the sheath 116 may include any number, pattern, configuration or size of bumps 140. In some embodiments, the sheath 116 may alternatively or additionally include bumps on its outer surface, to facilitate gripping the sheath 116 by the user. Other surface features may additionally or alternatively be used, such as grooves, ridges, striations or the like. In some embodiments, one or more bumps 140 or other surface features may be designed to help prevent the sheath from falling off the distal end 101 of the catheter 102.

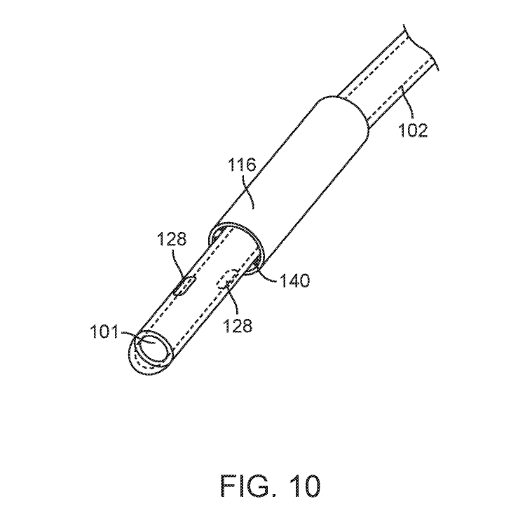

Referring to FIG. 10, a close-up view of the distal end 101 of the catheter 102 and the sheath 116 shows at least one of the bumps 140 inside the sheath 116.

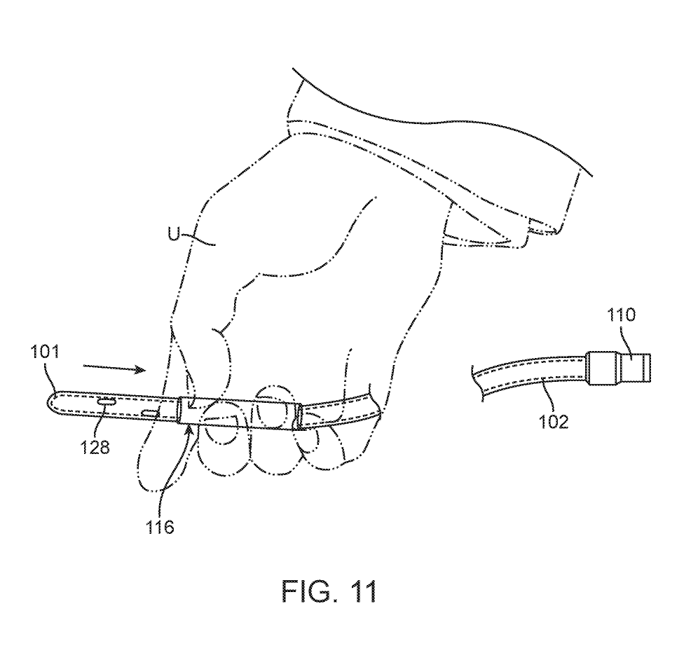

FIG. 11 shows a right hand of a user U, in the process of sliding the sheath 116 proximally along the catheter 102. This initial proximal movement of the sheath 116 is done before inserting the distal end 101 of the catheter 102 into the urethra. As shown here, the catheter 102 and sheath 116 may be manipulated with one hand.

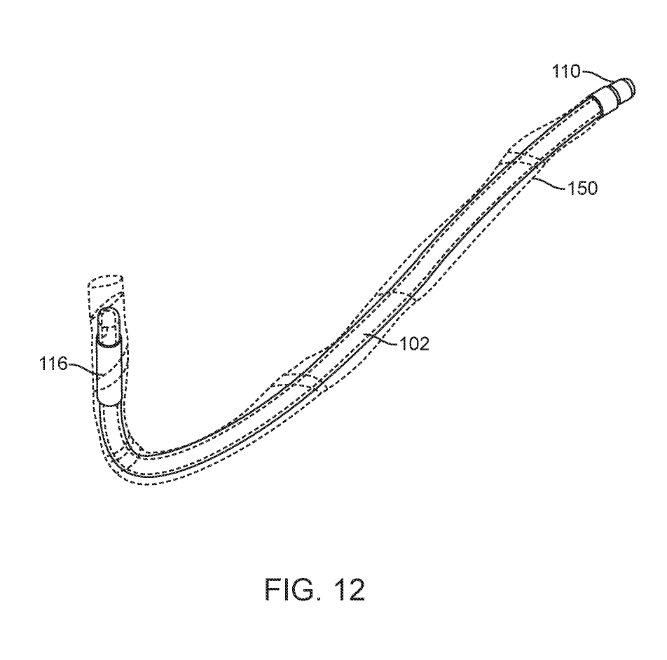

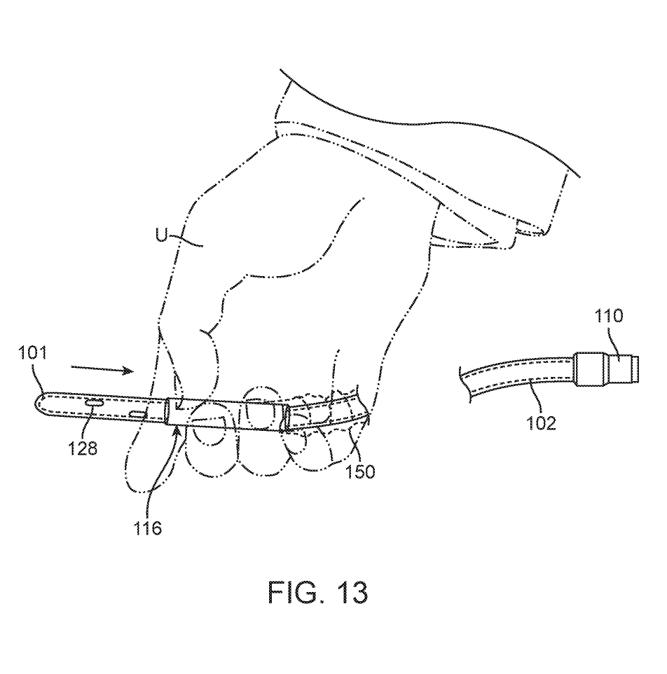

With reference to FIGS. 12 and 13, an optional outer sleeve 150 is illustrated. The sleeve 150 will be described in further detail below, but generally it may be attached to the proximal end 103 of the catheter 102 or to the adapter 110, and it may extend over the distal end 101 of the catheter 102, as illustrated. In an alternative embodiment, the distal end of the sleeve may be attached to the sheath 116. Referring to FIG. 13, as the sheath 116 is moved proximally along the catheter 102, for advancement of the catheter 102 into the urethra, the sleeve 116 is also retracted proximally, thus exposing the catheter 102. In this way, the catheter 102 may be covered and kept clean before it is advanced into the urethra. The sleeve 150 collapses on itself as it is pushed proximally during advancement of the catheter 102.

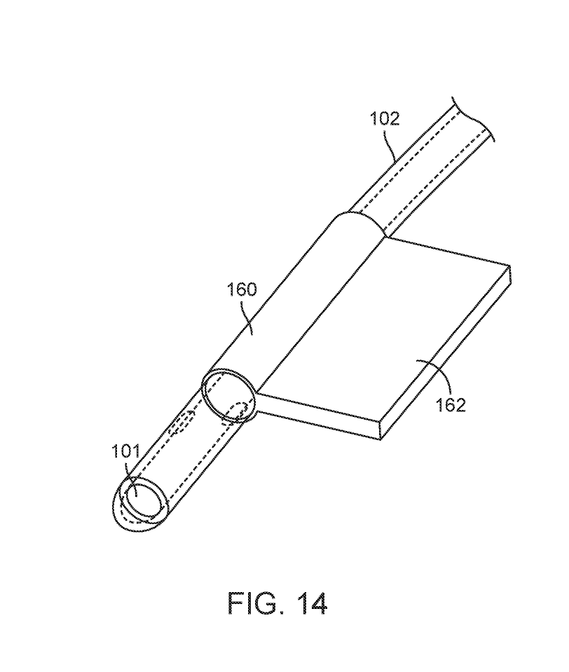

FIGS. 14-18 illustrate various alternative embodiments of a sheath for the catheter. In the embodiment illustrated in FIG. 14, the sheath 160 includes a straight, rectangular, lateral protrusion 162, which may act as a handle. This is only one example of such a protrusion 162, and alternative embodiments (some of which are illustrated in subsequent figures) may include any suitable number, size and/or shape of protrusion, made of any suitable material or combination of materials. The protrusion 162, which in this embodiment may be relatively rigid, may facilitate gripping and sliding of the sheath 160 along the catheter by the patient. The protrusion 162 may also help prevent the distal end of the sheath 160 from banging, abrading or otherwise harmfully contacting the outer opening of the urethra during initial insertion of the distal end 101 of the catheter into the urethra.

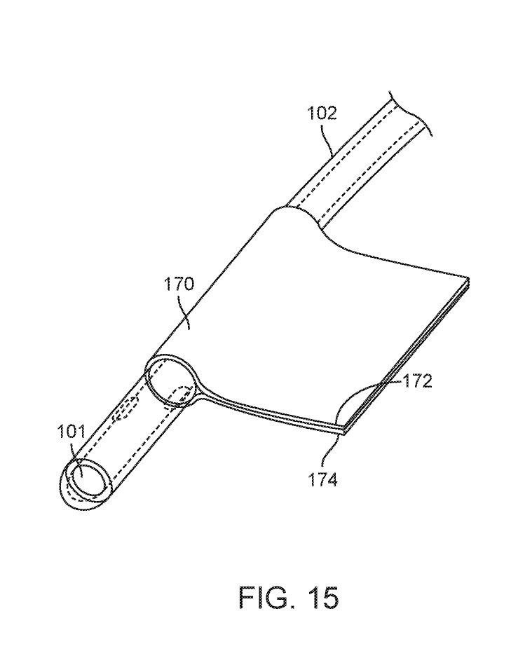

Referring to FIG. 15, in an alternative embodiment, a sheath 170 may be made of one piece of material that is wrapped around the catheter 102 and then attached to itself to form a flat protrusion having a top layer 172 and a bottom layer 174. This protrusion will provide similar benefits as the previously described embodiment. It may also be flexible, depending on the material used and the thickness of the layers 172, 174.

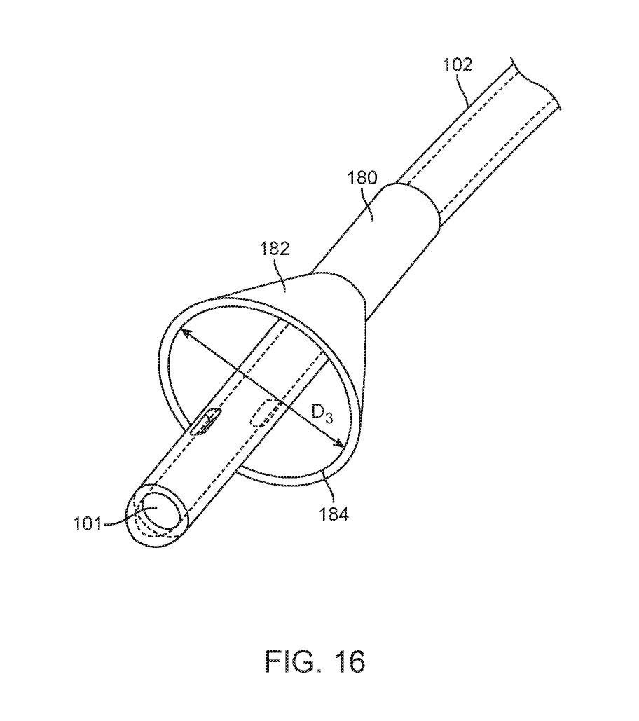

FIG. 16 illustrates yet another embodiment of a sheath 180, which includes a funnel-shaped protrusion 182. In this embodiment, the protrusion 182 has a wide, open end 184 with a diameter D3.

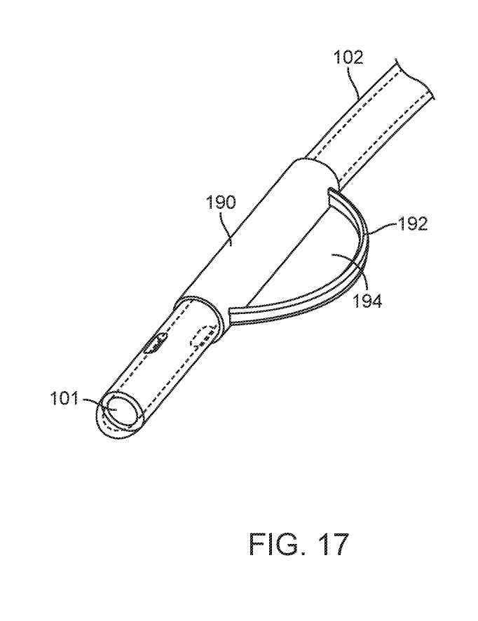

FIG. 17 illustrates another alternative embodiment of a sheath 190. In this embodiment, the sheath 190 includes a protrusion 192 made of a strip of material attached at opposite ends to one side of the sheath 190, such that the protrusion 192 bows out in a semi-circular shape to leave an opening 194. The patient may place a finger in the opening 194 for moving the sheath 190 along the catheter.

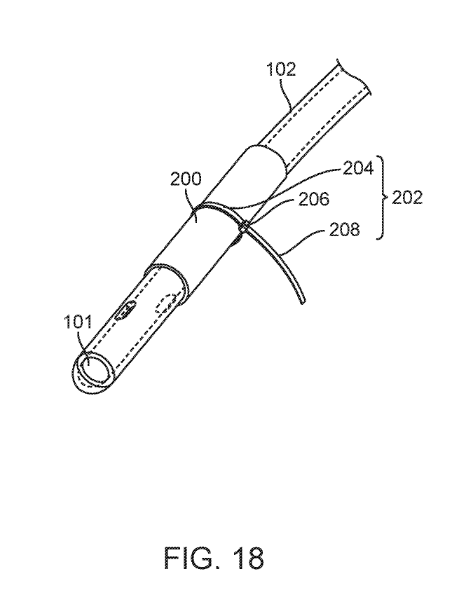

FIG. 18 shows another alternative embodiment of a sheath 200 for the catheter 102. In this embodiment, the protrusion 202 includes a tie 204 that is looped through a fastener 206 and has a free end 208 for grasping and moving the sheath 200. As with all previously described embodiments, the protrusion 202 in this embodiment may be made of any suitable material or combination of materials.

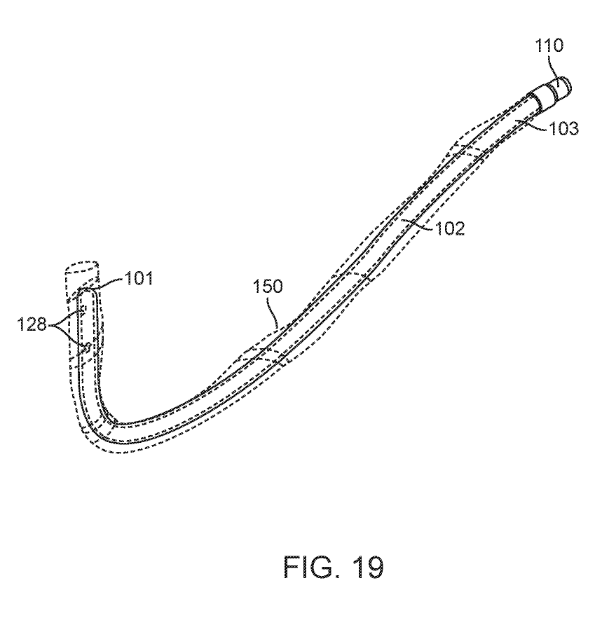

FIGS. 19-26 illustrate embodiments of the urinary catheter and enclosure system 100, and portions of the system 100, which include a flexible sleeve 150 for enhancing hygiene of the catheter 102. FIG. 19 shows the catheter 102, with the tubular adapter 110 on its proximal end 103 and the hygienic sleeve 150 extending from the tubular adapter 110 past the distal end 101 of the catheter. The sleeve 150 may be made of any suitable material, such as any suitable polymer or plastic. In FIG. 19 and several subsequent figures, the catheter 102 is shown without the sheath 116. However, any embodiments may also include the sheath 116, along with the sleeve 150, as will be described below.

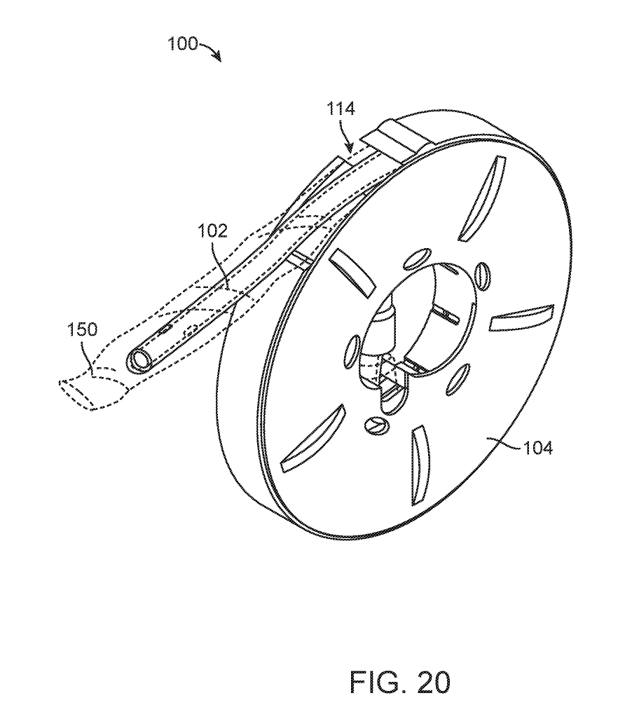

FIG. 20 illustrates the complete system 100, including the hygienic sleeve 150 but not including the urine collection bag 106. An alternative embodiment may include the bag 106.

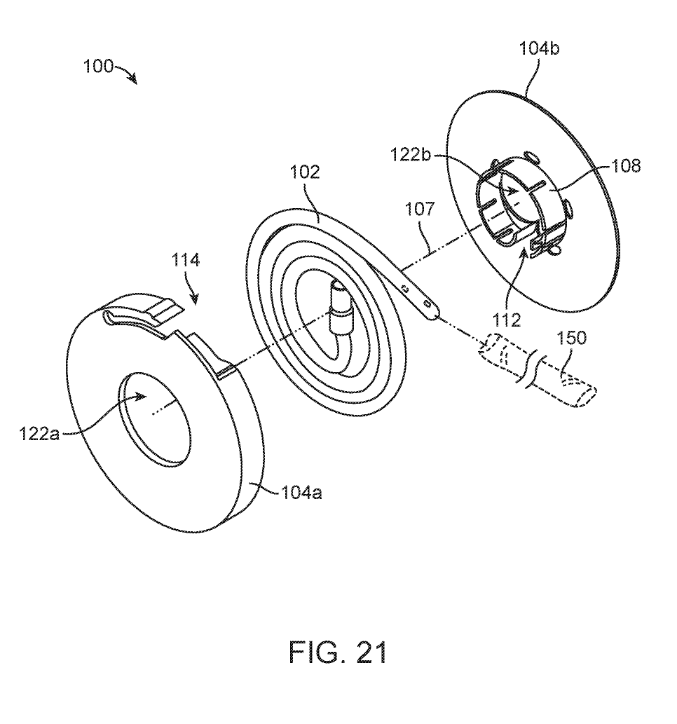

FIG. 21 is an exploded view of the urinary catheter and enclosure system 100, illustrating how the system 100 may be assembled. The sleeve 150 may be advanced over the catheter 102 at any point during assembly. As illustrated in this figure, a central axis of rotation 107 passes through the center of the opening 122 formed by the opening 122a in the top portion 104a of the enclosure and the opening 122b in the bottom portion 104b. To wind and unwind the catheter 102 into and out of the enclosure 104, one portion of the enclosure 104 is rotated around the central axis 107 relative to the other portion, as previously described.

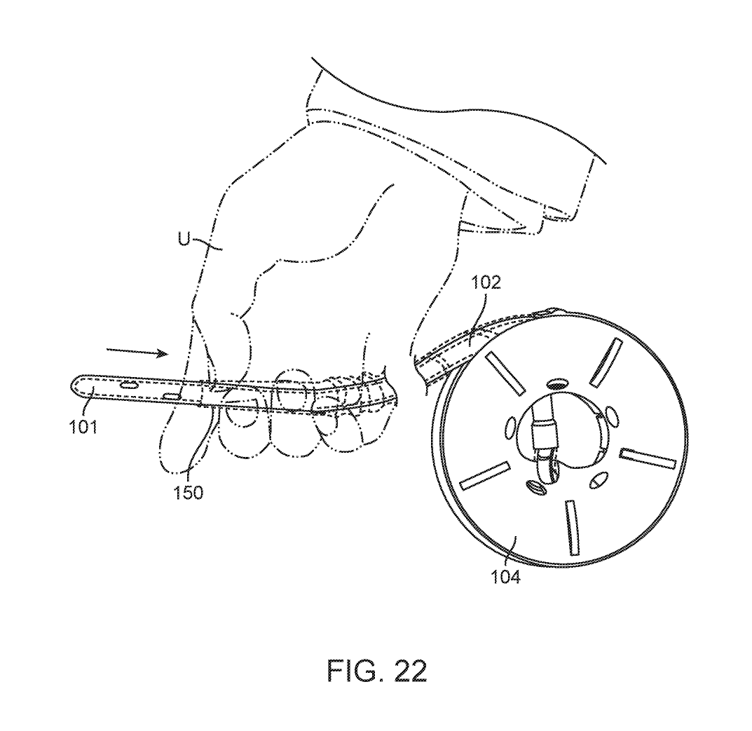

FIG. 22 illustrates a hand of a user U exposing the distal end 101 of the catheter 102, by retracting the sleeve 150. In alternative embodiments, this may be done by the same hand in conjunction with retracting the sheath 116.

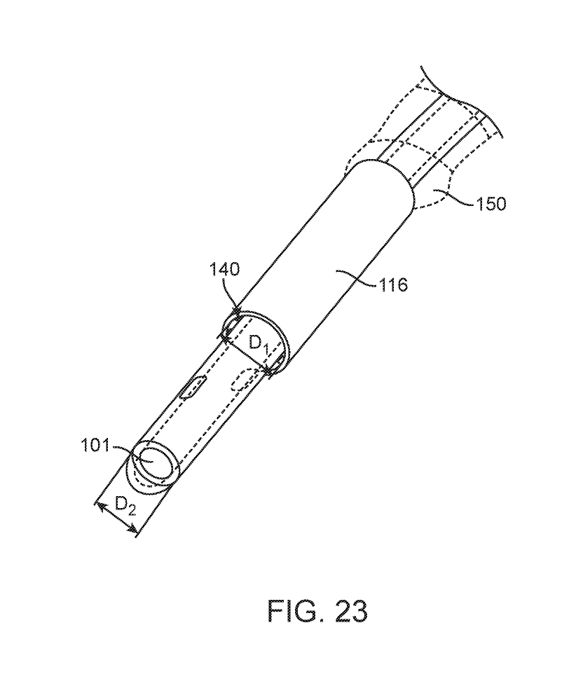

FIG. 23 illustrates a distal portion of the catheter 102, with the sheath 116 having internal protrusions 140 and the sleeve 150 attached to a proximal end of the sheath 116. This attachment to the proximal end of the sheath 116 is one embodiment, with an alternative embodiment being where the sleeve 150 extends over the sheath 116.

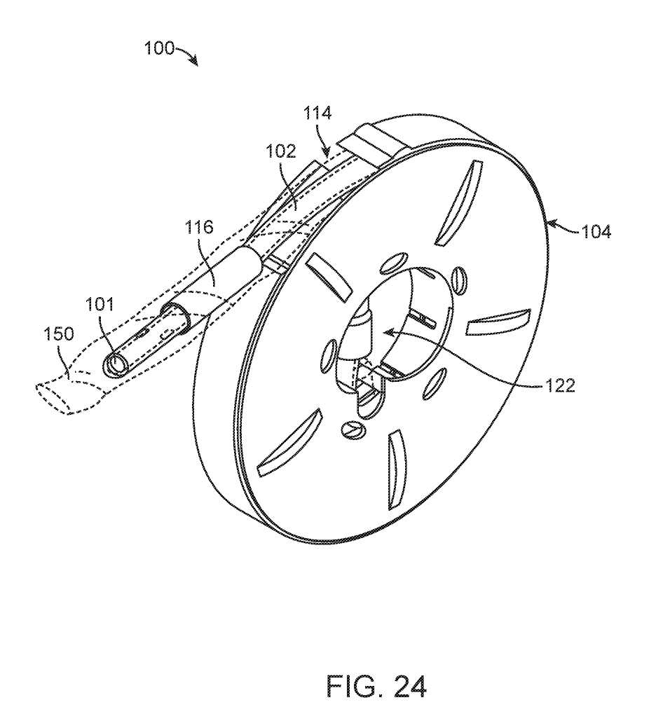

FIG. 24 illustrates the complete system 100, including the sheath 116 and the sleeve 150 extending over the sheath 116 and the distal end 101 of the catheter 102. Not shown here is the urine collection bag 106, which may additionally be included in some embodiments.

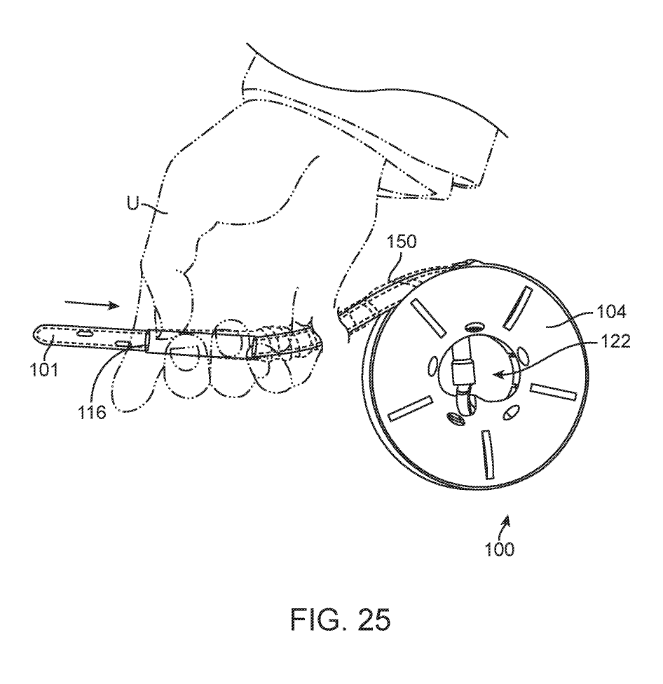

FIG. 25 is similar to FIG. 22, but in this embodiment the sleeve 150 and the sheath 116 are included and are being retracted by the user's hand.

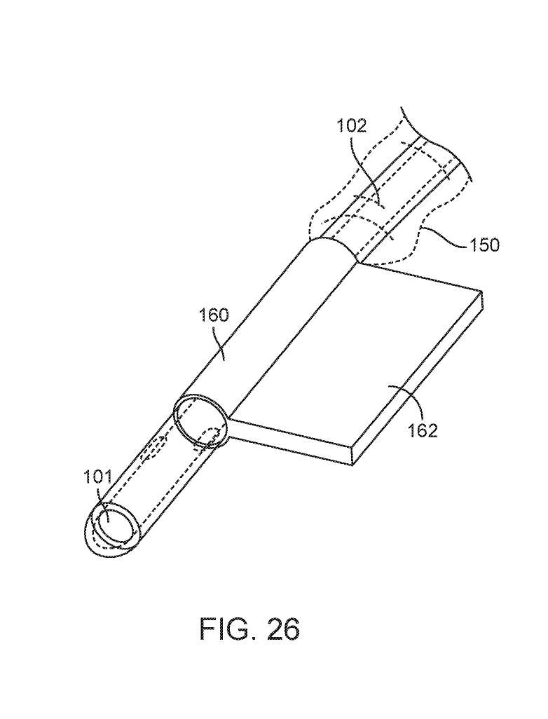

FIG. 26 illustrates one of the alternative embodiments of the sheath 160, with a protrusion 162, as described above. In this embodiment, however, the sleeve 150 is attached to a proximal end of the sheath 160. This is to illustrate that the sleeve 150 may be used with any of the described embodiments of the sheath 116, 160.

Any of the embodiments of sheaths with protrusions described above may be used along with the hygienic sleeve 150. In some embodiments, the protrusion(s) fit inside the sleeve 150. In other embodiments the sleeve 150 may include one or more openings to allow one or more sheath protrusions to extend through. Alternatively, the sleeve 150 may also include one or more pockets to accommodate the protrusion(s).

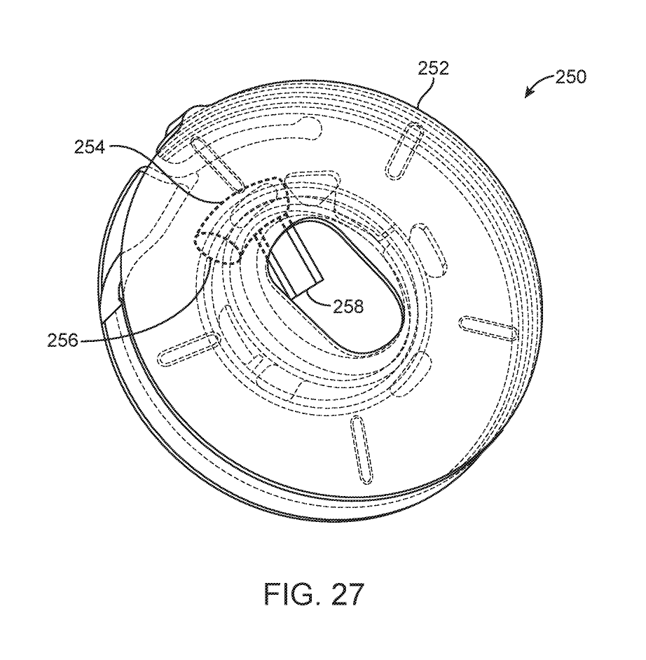

FIG. 27 is a perspective view of a urinary catheter and enclosure system 250, showing the enclosure 252 and highlighting an adapter 254. This adapter 254 is similar to other adapter embodiments described above, except that it includes a bend, thus forming approximately an L-shape. The adapter 254 has a catheter connecting end 256 for connecting to a proximal end of a urinary catheter (not shown), a bag connecting end 258 for connecting to a urine collection bag (also not shown), and a bend between the two ends 256, 258. Thus, a portion of the adapter 254 resides inside the enclosure 252, and a portion resides outside the enclosure 252, in the inner open area. The adapter 254 thus passes through the inner hole in the enclosure 252 to span from inside to outside of the enclosure 252. This shape for the adapter 254 may be advantageous, because it does not require the urinary catheter to make a bend to exit the enclosure 252, thus possibly preventing kinking of the urinary catheter.

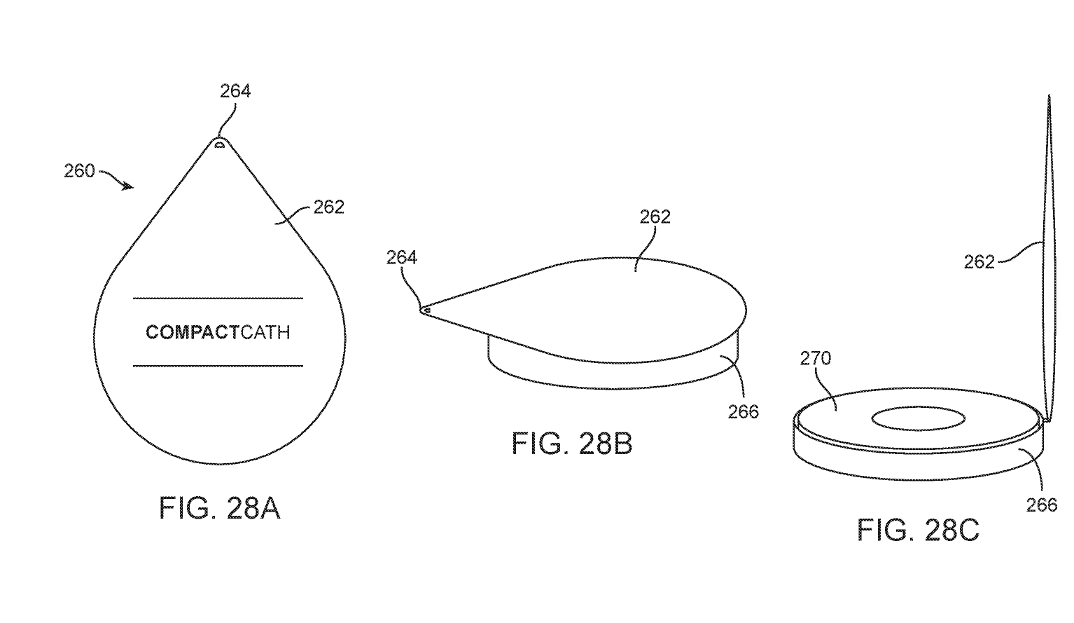

FIGS. 28A-28C are top and perspective views of a teardrop-shaped package 260 for containing a urinary catheter and enclosure system 270 before use. FIG. 28A is a top view, showing a top layer 262 and a peel tab portion 264 of the package 260. FIG. 28B is a perspective view, showing the top layer 262, peel tab portion 264, and a bottom container portion 266, which houses the urinary catheter and enclosure system 270. In FIG. 28C, the top layer 262 is peeled back to reveal the urinary catheter and enclosure system 270 within. In alternative embodiments, the package 260 may have any number of shapes, sizes and parts. The teardrop-shaped package 260, however, may be advantageous by way of its size and ease of use.

While the present disclosure has been described with reference to various embodiments, these embodiments are illustrative, and the scope of the disclosure is not limited to them. Many variations, modifications, additions, and improvements are possible. More generally, embodiments in accordance with the present disclosure have been described in the context of particular embodiments. Functionality may be separated or combined in procedures differently in various embodiments of the disclosure or described with different terminology. These and other variations, modifications, additions, and improvements may fall within the scope of the disclosure as defined in the claims that follow.

We claim:

1. A urinary catheter system, the system comprising:
a urinary catheter having a distal end configured for insertion into a urethra and a proximal end;
a sheath slidably disposed over a portion of the urinary catheter near the distal end and configured to allow the urinary catheter to be advanced into the urethra without directly touching the urinary catheter;
an enclosure for housing the urinary catheter, the enclosure comprising:
a bottom member comprising a base and an inner wall with an inner hole leading to an inner open portion of the enclosure; and
a top member comprising an outer wall with an outer hole leading out of the enclosure, wherein the top member is attached to the bottom member so as to rotate relative to the bottom member about an axis of rotation,
wherein rotating the top member or the bottom member relative to the other member causes the urinary catheter to wind into a spiral between the inner wall of the bottom member and the outer wall of the top member; and
a urine collection bag attached to the proximal end of the urinary catheter and housed within the inner open portion of the enclosure.

2. The system of claim 1, wherein an outer diameter of the enclosure is no greater than 60 millimeters.

3. The system of claim 1, wherein the inner wall of the bottom member of the enclosure comprises multiple curved, vertical slats, and wherein at least some of the vertical slats include a snap lock member for locking with a corresponding feature of the top member.

4. The system of claim 3, wherein two of the multiple vertical slats have a different curvature than the other multiple vertical slats, to form the inner hole in the inner wall.

5. The system of claim 1, wherein the outer hole in the outer wall of the top member of the enclosure is formed by:
an outer edge portion that is flexible to allow for pressing by the user to stop movement of the catheter into or out of the outer hole; and
an inner edge portion formed as a ramp to facilitate advancement of the catheter out of the outer hole.

6. The system of claim 1, further comprising an adapter coupled between the urine collection bag and the proximal end of the catheter, wherein the adapter allows the urine collection bag to be removed from the proximal end of the catheter and a new urine collection bag to be attached to the catheter.

7. The system of claim 6, wherein the adapter has a straight, tubular shape.

8. The system of claim 6, wherein the adapter comprises a bend.

9. The system of claim 1, wherein the sheath comprises at least one outward facing protrusion to facilitate gripping the sheath and sliding the sheath along the catheter by a user.

10. The system of claim 1, wherein the sheath comprises at least one inward facing protrusion to facilitate sliding the sheath along the catheter by a user.

11. The system of claim 1, further comprising a hygienic sleeve disposed over at least a portion of the catheter to prevent contamination of the catheter before and during insertion into the urethra.

12. The system of claim 11, wherein the hygienic sleeve comprises a material selected from the group consisting of plastics and polymers.

13. The system of claim 1, wherein the enclosure comprises a disposable package, and wherein the catheter, the sheath and the urine collection bag are completely removable from the enclosure.

14. The system of claim 1, further comprising a teardrop-shaped package for storing the urinary catheter, the sheath, the enclosure and the urine collection bag before use.

15. A method for using a urinary catheter system to void a bladder of a patient, the method comprising:
contacting, with one of the patient's hands, a sheath disposed over a urinary catheter near a distal end of the urinary catheter, wherein a majority of the urinary catheter is housed in an enclosure comprising a top member and a bottom member, and wherein the distal end of the urinary catheter is located outside an outer hole in the enclosure;
advancing the urinary catheter out of the enclosure by pulling on the urinary catheter via the sheath;
advancing the distal end of the urinary catheter into a urethra of the patient while continuing to contact only the sheath and not the urinary catheter with the patient's hand;
sliding the sheath proximally along the urinary catheter;
advancing the distal end of the urinary catheter through the patient's urethra and into the patient's bladder, using the sheath;
unfolding a urine collection bag attached to a proximal end of the urinary catheter, wherein the urine collection bag is stored in an inner open portion of the enclosure before unfolding; and
voiding the patient's bladder through the catheter, so that urine passes from the patient's bladder through the urinary catheter and into the urine collection bag.

16. The method of claim 15, further comprising, after voiding the patient's bladder:
removing the urine collection bag from the urinary catheter; and
winding the urinary catheter into the enclosure by twisting at least one of a top portion of the enclosure or a bottom portion of the enclosure.

17. The method of claim 16, further comprising attaching a new urine collection bag to the proximal end of the urinary catheter via a tubular adapter coupled between the proximal end of the urinary catheter and the new urine collection bag.

18. The method of claim 15, further comprising removing the urinary catheter and the urine bag completely from the enclosure.

19. The method of claim 18, further comprising disposing of the urinary catheter, the enclosure and the urine bag after the bladder is voided.

20. The method of claim 15, wherein contacting the sheath comprises contacting at least one outward facing protrusion on the sheath.

21. The method of claim 15, wherein sliding the sheath proximally along the urinary catheter is performed multiple times as the catheter is advanced through the urethra.

22. The method of claim 15, further comprising sliding a hygienic sleeve proximally along the urinary catheter during advancement of the catheter through the urethra.
